(19) United States
(12) Patent Application Publication
(10) Pub. No.: US 2022/0339263 A1
(43) Pub. Date: Oct. 27, 2022

(54) COMPOSITION FOR TREATING CANCER EXPRESSING SPARC

(71) Applicants: SEOUL NATIONAL UNIVERSITY R&DB FOUNDATION, Seoul (KR); SEOUL NATIONAL UNIVERSITY HOSPITAL, Seoul (KR)

(72) Inventors: Keon Wook KANG, Seoul (KR); Myung Geun SONG, Seoul (KR); Cho Rong PARK, Seoul (KR); Yun-Sang LEE, Gyeonggi-do (KR); Hye Won YOUN, Seoul (KR); Ji Yong PARK, Seoul (KR); Se Ra OH, Seoul (KR)

(21) Appl. No.: 17/677,162

(22) Filed: Feb. 22, 2022

(30) Foreign Application Priority Data
Feb. 19, 2021 (KR) 10-2021-0022433

(51) Int. Cl. A61K 38/38; A61P 35/00; A61P 25/00

(52) U.S. Cl. CPC A61K 38/38; A61P 35/00; A61P 25/00

(57) ABSTRACT

A composition for treatment of a cancer according to an embodiment of the present disclosure expresses secreted protein acidic and rich in cysteine (SPARC). The composition includes an albumin and at least one cysteine bound thereto.

### CROSS-REFERENCE TO RELATED APPLICATION(S)

This application claims the benefit under 35 USC § 119(a) of Korean patent Application No. 10-2021-0022433, filed on Feb. 19, 2021, in the Korean Intellectual Property Office, the entire disclosure of which is incorporated herein by reference for all purposes.

### BACKGROUND

### 1. Field of the Invention

The present invention relates to a composition for treatment of a cancer expressing SPARC.

### 2. Description of the Related Art

A current standard treatment method of glioma is surgical excision. However, all types of gliomas are sometimes present in functional brain regions, thus it is difficult to completely remove the same.

In general, anti-cancer chemotherapy induces toxic side-effects for a human body, and is a high-cost therapeutic method which requires a great expense in an economic aspect. Therefore, development for designing formulations to reduce side effects while maximizing medical efficacy has been proceeded, and a drug-protein composite corresponds to a representative form thereof.

Cisplatin is a platinum-based drug and is known to be effective for anti-cancer response, however, entails problems due to cytotoxicity. Further, this drug cannot penetrate a brain-barrier and thus is not useful for treatment of a brain tumor. In addition, CDDP bound to HSA (HSA-CDDP) is known to be non-active in a therapeutic aspect.

Moreover, although SPARC has high expression in glioma which is an albumin-bonded protein, studies on suitability of the drug carrier HSA as a therapeutic agent for the glioma have not been sufficiently executed.

### SUMMARY

It is an object of the present invention to provide a composition for treatment of a cancer expressing secreted protein acidic and rich in cysteine (SPARC).

To achieve the above object, the following technical solutions are adopted in the present invention.

1. A composition for treatment of a cancer expressing secreted protein acidic and rich in cysteine (SPARC), including an albumin and at least one cisplatin bound thereto.

2. The composition for treatment of a cancer according to the above 1, wherein 1 to 8 cisplatins are bound to the albumin.

3. The composition for treatment of a cancer according to the above 1, wherein 4 to 5 cisplatins are bound to the albumin.

4. The composition for treatment of a cancer according to the above 3, wherein a conjugate of the albumin and the 4 to 5 cisplatins bound thereto has a molecular weight of 65,000 to 70,000 Da.

5. The composition for treatment of a cancer according to the above 3, wherein a conjugate of the albumin and the 4 to 5 cisplatins bound thereto has a size of 5 nm to 100 nm.

6. The composition for treatment of a cancer according to the above 1, wherein the cisplatin is bound to His side-chain residue or Met side-chain residue on a surface of the albumin.

7. The composition for treatment of a cancer according to the above 6, wherein His105 or His288 on the surface of the albumin is bound to the cisplatin.

8. The composition for treatment of a cancer according to the above 6, wherein Met298, Met329 or Met548 on the surface of the albumin is bound to the cisplatin.

9. The composition for treatment of a cancer according to the above 6, wherein the binding is obtained by a coordinate bond.

10. The composition for treatment of a cancer according to the above 1, wherein the cancer expressing SPARC is at least one selected from the group consisting of a brain tumor, melanoma, breast cancer, rectal cancer and stomach cancer.

11. The composition for treatment of a cancer according to the above 10, wherein the cancer is a brain tumor.

When using the composition of the present invention, it is possible to improve a blood circulation time of an anti-cancer treatment drug.

When using the composition of the present invention, it is possible to reduce cytotoxicity and renal toxicity of the anti-cancer treatment drug.

The composition of the present invention may have excellent targeting function for a cancer to express SPARC, whereby the anti-cancer treatment drug may exhibit excellent cancer cell accumulation effects.

### BRIEF DESCRIPTION OF THE DRAWINGS

FIG. 1 shows results of cell uptake by HSA-CDDP (human serum albumin-cisplatin), more particularly, HSA-CDDP(4.1). Specifically, (a) of FIG. 1 is representative images of cellular uptake in a cell. In order to observe dependency of SPARC in HSA-CDDP absorption, exogenous secreted protein acidic and rich in cysteine (SPARC) was treated (scale bars, 50 μm), (b) of FIG. 1 illustrates quantification of cell uptake of FNR648-HSA in a cell, and (c) of FIG. 1 illustrates quantification of cell uptake in FNR648-HSA-CDDP cell. Data is indicated by average SD (n=5). ***p<0.001;

FIG. 2 illustrates cytotoxicity of CDDP and HSA-CDDP in U87MG and U87MG-shSPARC cells. Specifically, (a) of FIG. 2 illustrates cytotoxicity of CDDP in U87MG and U87MG-shSPARC cells, (b) of FIG. 2 illustrates cytotoxicity of HSA-CDDP(4.1) in U87MG and U87MG-shSPARC cells (n=5 in each concentration). Each compound was subjected to treatment for 72 hours, and (c) of FIG. 2 illustrates cytotoxicity of each of HSA-CDDP(1.5), HSA-CDDP(2.3), HSA-CDDP(5.4) and HSA-CDDP(7.7) when varying the number of CDDPs bound to HSA in U87MG cells (n=6 in each concentration);

FIG. 3 illustrates results of confirming cytotoxicity of HSA-CDDP, more particularly, HSA-CDDP(4.1). CDDP and HSA-CDDP(4.1) were subjected to analysis of cell apoptosis. Cells were subjected to treatment with CDDP or HSA-CDDP(4.1) for 72 hours. Each concentration was based on 30 to 40% of living cells in the result of cck-8 of FIG. 2. The numbers in red were based on 30 to 40% of living cells in the result of cck-8 of FIG. 2. The number in red means a ratio of cells under total cell apoptosis in each group, which is the sum of numbers of smaller letters in each figure;

FIG. 4 illustrates results of analyzing cell apoptosis of CDDP and HSA-CDDP in U87MG and U87MG-shSPARC cells. Specifically, (a) and (b) of FIG. 4 show results of treating cells with CDDP and HSA-CDDP, more particularly, HSA-CDDP(4.1) at different concentrations, respectively;

FIG. 5 illustrates anti-tumor effects of HSA-CDDP, more particularly, HSA-CDDP(4.1) in a xenograft tumor model. Specifically, (a) and (b) of FIG. 5 show tumor volumes of U87MG and U87MG-shSPARC, respectively. Further, (c) and (d) of FIG. 5 show weights of U87MG and U87MG-shSPARC in a mouse xenograft tumor model, respectively. Further, (e)and (f) of FIG. 5 show Kaplan-Meier viability curves in regard to U87MG and U87MG-shSPARC in the tumor model, respectively. Data is indicated by average SD. ***p <0.001;

FIG. 6 illustrates results of analyzing in vivo distribution of HSA-CDDP, more particularly, HSA-CDDP(4.1) in mouse by ICP-MS. CDDP or HSA-CDDP was treated in the same treatment way (on alternate days, seven times). Specifically, (a) of FIG. 6 shows CDDP accumulation in organs and blood, wherein the left bar of each item in the graph shows CDDP while HSA-CDDP is shown in the right bar, and (b) of FIG. 6 shows CDDP accumulation in tumor (% ID; percent injected dose). All organs and tumors were collected 72 hours after final drug treatment. Data is indicated by average SD (n=4). *p<0.05;

FIG. 7 illustrates results of analyzing biological safety of HSA-CDDP, more particularly, HSA-CDDP(4.1) in mouse. Specifically, (a) of FIG. 7 shows weights and serum concentrations of aspartate transaminase (AST), alanine aminotransferase (ALT), blood urea nitrogen (BUN) and creatinine, respectively, and (b) of FIG. 7 shows images of stained organs in kidney, liver and pancreas using hematoxylin-eosin (H&E). In the kidney of a mouse treated with CDDP, degeneration of renal tubules (fine arrow) and vacuolation of epithelial cells (bold arrow) were observed (Scale bars, 100 μm for kidney and liver, and 200 μm for pancreas);

FIG. 8 shows TUNEL analysis images of renal tissues. Specifically, (a) of FIG. 8 is images of 200× (scale bars, 100 μm), and (b) of FIG. 8 is images of 400× (scale bars, 50 μm); and

FIG. 9 illustrates images of serum stability and in vivo distribution of HSA-CDDP, more particularly, HSA-CDDP(4.1). Specifically, (a) of FIG. 9 shows serum stability in vitro HSA-CDDP, and (b) of FIG. 9 shows SPECT image of [¹⁷⁷Lu]-labeled HSA-CDDP(4.1) in a xenograft tumor model.

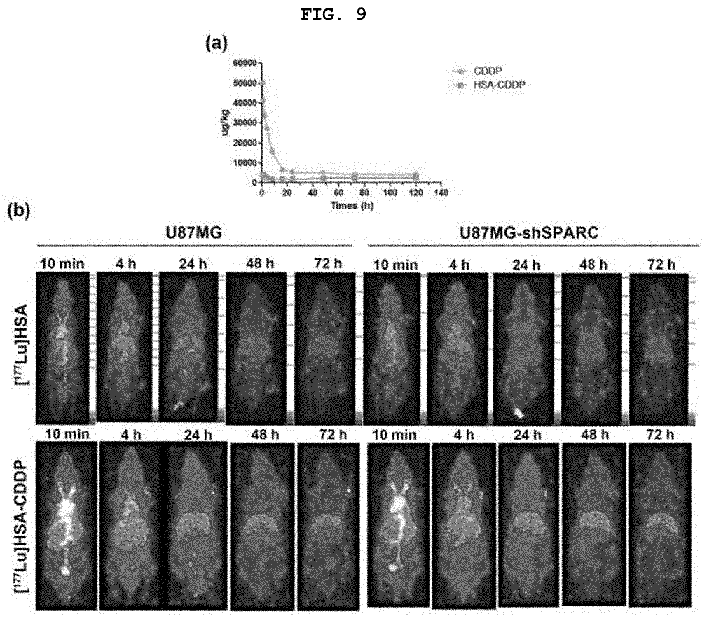

### DETAILED DESCRIPTION

The present invention provides a composition for treatment of a cancer expressing secreted protein acidic and rich in cysteine (SPARC), which includes an albumin and at least one cisplatin bound thereto.

Albumin is a simple protein having a spherical shape, which is rich in plasma or the like. Specifically, the albumin present in the plasma refers to plasma albumin. Albumin is broadly distributed in bio-cells or body fluid. In particular, plasma albumin constitutes a basic material of cell and plasma together with globulin of the plasma. Albumin counts about 50 to 60% of proteins constituting a human body, and may play important roles such as an adjustment of a water amount of the blood and body through osmotic regulation.

Among albumins, a human serum albumin (HSA) may have an important role in delivery and arrangement of endogenous and exogeneous ligands in the blood.

In the present invention, the albumin includes a SPARC binding site and a cisplatin binding site, and therefore, it may be bound with SPARC and cisplatin. In this case, the albumin does not affect binding at other binding sites, by the binding of the SPARC or cisplatin.

Cisplatin (cis-diammine dichloroplatinum (II), CDDP) is one of chemotherapic drugs widely used for solid cancers. Cisplatin may be bound to the human serum albumin (HSA) which is a plasma protein most abundant in the serum.

The secreted protein acidic and rich in cysteine (SPARC) is a protein possibly bound with an albumin highly expressed in some cancers, and may function to control cell-matrix interaction, proliferation, survival and movement.

According to the present invention, a conjugate of albumin and drug may be an albumin-cisplatin conjugate, and for example, may be HSA-CDDP (human serum albumin-cisplatin) conjugate.

According to the present invention, the albumin or conjugate of albumin and drug may be bound with a protein (for example, glycoprotein 60 (GP60)) present in a blood-brain barrier (BBB) surface.

According to the present invention, a conjugate of albumin and cisplatin bound thereto may pass through the blood-brain barrier (BBB).

According to the present invention, the albumin or conjugate of albumin and drug passes through the BBB, and then may target a SPARC protein, in which brain tumor cells contained therein are over-expressed, thus to be bound with the above protein.

According to the present invention, the albumin or conjugate of albumin and drug may be accumulated in a tumor by SPARC-mediated passive targeting and active targeting. For example, the albumin or conjugate of albumin and drug may be accumulated in the tumor due to enhanced permeability and retention (EPR) effects.

According to the present invention, a composition including an albumin or conjugate of albumin and drug may be used for targeted therapy of a cancer expressing SPARC. For example, an HSA-CDDP conjugate may be used for removing glioma residue in the brain.

According to the present invention, the HSA-CDDP conjugate may be absorbed into cells by endocytosis. Further, the HSA-CDDP conjugate is degraded in the cells, which in turn can be discharged in free CDDP state from HSA.

According to the present invention, when forming the HSA-CDDP conjugate, excretion and renal accumulation of CDDP meditated by OCT2 may be reduced.

According to the present invention, the albumin may be bound with at least one cisplatin. For example, 1 to 10, 1 to 8, 1 to 7, 1 to 5 or 1 to 4 cisplatins may be bound to the albumin, but it is not limited thereto.

In an embodiment of the present invention, HSA-CDDP conjugates including cisplatins having the average molecular numbers of 1.5, 2.3, 4.1, 5.4 and 7.7 moles, respectively, bound to 1 mole of albumin (abbrev. to HSA-CDDP(1.5), HSA-CDDP(2.3), HSA-CDDP(4.1), HSA-CDDP(5.4), HSA-CDDP(7.7), respectively, in the present specification) were prepared. It was confirmed that toxicity to U87MG cell line becomes higher with increase in the number of cisplatins bound to albumin.

According to the present invention, 4 to 5 cisplatins may be bound to one albumin, and the albumin may be a human serum albumin.

According to the present invention, the conjugate of albumin and cisplatin bound thereto may have a molecular weight of 65,000 to 70,000 Da, for example, 66,500 to 68,000 Da.

According to the present invention, the conjugate of albumin and cisplatin bound thereto may have a size of 5 to 100 nm, for example, 7 to 10 nm.

A binding site of cisplatin to the albumin is not particularly limited, but may be His side-chain residue or Met side-chain residue.

According to the present invention, the cisplatin may be bound to His105, His288, Met298, Met329 or Met548 side-chain residue on the surface of the albumin.

According to the present invention, binding CDDP to HSA may be irreversible, for example, the cisplatin and the albumin may form a conjugate through a coordinate bond.

The composition of the present invention includes the cisplatin, which is commonly known to have anti-cancer effects. Therefore, all types of cancers to which the cisplatin exhibits medical efficacy may correspond to the cancers as subjects to be treated.

With regard to the composition of the present invention, the cisplatin is bound to the albumin to form a conjugate, and this conjugate may be bound to SPARC expressed by cancer cells or tissues, thereby targeting the cancer. Therefore, when using the composition of the present invention in a cancer expressing SPARC, more useful effects may be achieved.

Subjects to which the composition of the present invention is applied, that is, cancers expressing SPARC may include brain tumor, melanoma, breast cancer, rectal cancer or stomach cancer. In addition, all types of cancers known to express SPARC may be included in the above subjects. According to the present invention, the cancer expressing SPARC may include brain tumor. According to the present invention, SPARC may be expressed in glioma, melanoma or breast cancer at a high concentration.

According to the present invention, the brain tumor may include glioma, for example, astrocytoma, glioblastoma or oligodendroblastoma, but it is not limited thereto.

According to the present invention, astrocytic tumor (that is, “astrocytoma”) may occur in astrocytes and an invasive tumor with an unclear barrier to surrounding normal tissues. The astrocytoma is very diverse in terms of sites of occurrence, age, growth rate, invasiveness, morphological shape, clinical course and the like.

The astrocytoma may be classified from stage 1 of positive tumor to stage 4 of the most malignant tumor according to a degree of malignancy.

Stage 1: Pilocytic astrocytoma

Stage 2: Diffuse astrocytoma

Stage 3: Anaplastic astrocytoma

Stage 4: Glioblastoma

Since it is known that, as the severity of the brain tumor is increased, an extent of expressing SPARC is increased (for example, a concentration of SPARC is increased around brain tumor cells), the composition of the present invention may exhibit higher targeting ability than the case of applying to a brain tumor with increased severity.

According to the present invention, oligodendroglial tumor (“oligodendroblastoma”) is a tumor occurring in oligodendrocytes (or oligodendroglia). According to the present invention, the oligodendroblastoma may be a mixed glioma form blended with astrocytoma.

According to the present invention, cells in which the brain tumor is formed may be glial cells, and the glial cells may include astrocytes, microglias, oligodendroglias, ependymal cells, Schwann's cells or capsular cells, but it is not limited thereto.

### EXAMPLES

### 1.Materials and Methods

### 1.1. Cell Lines

Human glioma cells (U87MG) were obtained from American Type Culture Collection (ATCC; Manassas, Va., USA). Low SPARC-expressing U87MG cells, U87MG-shSPARC, were established from a previous study.

Cells were grown in Minimum Essential Medium (MEM; Gibco, Grand Island, N.Y., USA) supplemented with 10% heat-inactivated fetal bovine serum (FBS; Gibco, Grand Island, N.Y., USA) and 1% antibiotics containing penicillin/streptomycin (Invitrogen, Grand Island, N.Y., USA). All cells were maintained at 37° C. in a humidified atmosphere with 5% CO2.

### 1.2. Preparation of CDDP Conjugated HSA

(1) Human serum albumin (MP biomedicals, Irvine, Calif., USA) was dissolved in PBS as 22 mg/mL concentration. CDDP (Sigma-Aldrich, St. Louis, Mo., USA) was dissolved in PBS as a 1 mg/mL concentration.

HSA and CDDP solutions were added to a new bottle at a molar ratio of 1:10 (1:1 volume ratio) and stirred for 24 h at 37° C.

To remove unconjugated CDDP and concentrate HSA-CDDP, centrifugal filtration was conducted using an Amicon Ultra centrifugal filter unit (nominal molecular weight limit 30 kDa; Millipore, Burlington, Mass., USA).

The HSA-CDDP concentration was measured with a bicinchoninic acid (BCA) protein assay kit (Pierce Endogen, Rockford, Ill., USA), and the molecular weight was analyzed by matrix-assisted laser desorption/ionization-time of flight (MALDI-TOF) using the TOF-TOF 5800 System (AB SCIEX, Framingham, Mass., USA) to check the amount of CDDP per HSA in HSA-CDDP.

(2) Four (4) types of HSA-CDDPs were prepared to evaluate cytotoxicity relative to the number of CDDPs bound to HAS. HSA and CDDP solutions were added at a molar ratio of 1:2.5 to 20. The average numbers of molecules of CDDPs bound to one mole of HSA were 1.5, 2.3, 5.4 and 7.7 moles, respectively.

### 1.3. Conjugation of Fluorescence Dye to HSA and HSA-CDDP

We conjugated HSA with FNR648 fluorescence dye following the procedure described in our previous publication. For FNR648 dye labeling to HSA-CDDP, we used the same method as that used for HSA. First, HSA-CDDP was modified using dibenzocyclooctyne (DBCO)-NHS. DBCO-HSA-CDDP was reacted with FRN648 dye at a molar ratio of 1:1 for 30 min at 37r. Fluorescence labeled HSA-CDDP was purified using PD-10 columns (GE Healthcare, Buckinghamshire, UK) and eluted with PBS.

### 1.4. Confocal Microscopy Imaging for Cellular Uptake of FNR648-HSA-CDDP

Cellular uptake of fluorescence labeled HAS or HSA-CDDP(4.1) followed the same procedure described in our previous publication. We incubated cells with FNR648-HSA or FNR648-HSA-CDDP for 2 h at 37° C. In the exogenous SPARC treatment group, human SPARC (5 μg/mL) and each compound(FNR648-HSA or FNR648-HSA-CDDP) were co-incubated for 2 h at 37° C. Five randomized images were acquired from all tests to quantify FNR648-HSA or FNR648-HSA-CDDP uptake by the cells. In each group, the average signal intensity of FNR648-HSA or FNR648-HSA-CDDP was divided by the number of DAPI-positive cells, which represented the number of viable cells in the image. This ratio was considered as cellular uptake of each group and used to quantify cellular uptake of each material.

### 1.5. Cellular Cytotoxicity Studies

(1) U87MG and U87MG-shSPARC cells were added to 96-well plates (4000 cells/well). After overnight incubation, the cells were incubated with CDDP, HSA-CDDP at different concentrations for 72 h. Cell viability was analyzed using a Cell Counting Kit-(CCK-8, Dojindo Molecular Technologies, Tokyo, Japan) assay according to the manufacturer's protocols.

(2) To execute cellular cytotoxicity experiments of HSA bound with CDDPs in different numbers, U87MG cells were added to a 96-well plate (4000 cells/well) and incubated overnight. Then, the cells were incubated with HSA-CDDP(1.5), HSA-CDDP(2.3), HSA-CDDP(5.4) and HSA-CDDP(7.7) at different concentrations, respectively, for 72 hours. Cell viability was analyzed using a Cell Counting Kit-8 (CCK-8, Dojindo Molecular Technologies, Tokyo, Japan) assay according to the manufacturer's protocols.

### 1.6. Cell Apoptosis Study

U87MG and U87MG-shSPARC cells were placed into 6-well plates (1.2×10⁵ cells/well). After overnight incubation, cells were treated with CDDP, HSA-CDDP for 72 h. Cells were then harvested and co-stained with PI and Annexin V using an Annexin V-FITC apoptosis detection kit (BD, San Jose, Calif., USA). The apoptosis of cells was analyzed using flow cytometry.

### 1.7. Animal Xenografts Tumor Model and Anti-Tumor Effect In Vivo

All animal studies were performed under approval from the Seoul National University Institutional Animal Care and Use Committee (IACUC No. 18-0231, 1 November 2018). BALB/c nude mice (5-week-old, male) were purchased from Orient Bio Inc. (Seongnam, Korea). U87MG or U87MG-shSPARC cells (2×10⁶ cells/site) were injected subcutaneously into the right lower flanks.

When tumor volume approached 50 mm³, mice were randomly divided into 3 groups (U87MG: n=12 for PBS group, n=9 for CDDP group and n=11 for HSA-CDDP group. U87MG-shSPARC: n=7 for PBS group, n=6 for CDDP group and n=9 for HSA-CDDP group).

Mice were IV administrated (7 times, every other day) with PBS, CDDP, or HSA-CDDP(4.1). The drug dose of CDDP was 3 mg/kg and the dose for HSA-CDDP was the equivalent amount of CDDP (3 mg/kg CDDP from HSA-CDDP).

The tumor size and body weight of each mouse were recorded every other day. Tumor volume was calculated using the equation V=0.5×L×W², where L represents tumor length and W represents tumor width.

To evaluate the survival rate of CDDP and HSA-CDDP in the tumor model, mice were monitored and euthanized following the humane endpoints guideline (specifically, rapid weight loss of 15-20% within a few days or tumor volume is larger than 2000 mm³) .

### 1.8. Biodistribution of CDDP Using ICP-MS

Normal mice were divided into 2 groups (n=4 for each group) and IV administrated with CDDP or HSA-CDDP(4.1) at the same dose and time schedule as the anti-tumor effect examination (3 mg/kg CDDP and equivalent dose of HSA-CDDP as CDDP every other day, seven times). At 72 h after the final IV administration (15 days after first IV administration), mice were euthanized and organs (brain, heart, liver, kidney, spleen, and lung) and blood were acquired and weighed.

For tumor CDDP distribution, U87MG or U87MG-shSPARC cells (2×10⁶ cells/site) were injected subcutaneously into the right lower flanks. When tumor volume approached 50 mm³, mice were randomly divided into 2 groups (n=5 for each group) and IV administrated CDDP or HSA-CDDP(4.1) (every other day, seven times). At 72 h after the last IV administration (15 days after first administration), mice were euthanized, and the tumor was acquired.

All samples (organs, blood, and tumors) were lyophilized and we analyzed the amount of CDDP using the ICP-MS (NexION 350; Perkin-Elmer, Waltham, Mass., USA) installed at the National Center for Inter-university Research Facilities (NCIRF) at Seoul National University.

### 1.9. Hematology Analysis

Mice were treated with PBS, CDDP, or HSA-CDDP (n=4 for each group) as the therapy treatment (every other day, seven times) and murine blood samples were acquired at 72 h after the final IV administration (15 days after first treatment). After centrifugation at 4° C. for 20 min, the plasma was collected for blood biochemical analysis. The concentrations of AST, ALT, BUN, and creatinine were analyzed at DKKorea (Seoul, Korea).

### 1.10. Histopathology Examination

The kidney, liver, and spleen of the drug-treated mice were acquired and fixed with 4% paraformaldehyde. Tissues were embedded in paraffin and stained with hematoxylin and eosin (H&E). H&E staining images were acquired using an optical microscope (Olympus BX43, Tokyo, Japan).

### 1.11. Serum Stability of HSA-CDDP

The CDDP stability of HSA-CDDP in serum was examined following method in another publication. Serum albumin/HSA-CDDP solution were incubated at 37° C. and free CDDP in serum was measured at 0.5, 1, 2, 4, 8, 16, 24, 48, 72 and 120 h after incubation. To obtain free CDDP from serum, an Amicon Ultra centrifugal filter unit (nominal molecular weight limit 30 kDa; Millipore, Burlington, Mass., USA) was used. The CDDP concentration measured in the supernatant (bottom of the tube, smaller than 30 KDa) corresponded to the free CDDP level. The concentration of CDDP in the supernatant was analyzed using the ICP-MS (NexION 350; PerkinElmer, Waltham, Mass., USA) installed at the National Center for Inter-university Research Facilities (NCIRF) at Seoul National University.

### 1.12. SPECT Imaging

Small animal SPECT imaging of tumor-bearing mice was performed using Nano SPECT/CT plus (Mediso medical imaging system, Budapest, Hungary). Mice were injected with 18.5 MBq of ¹⁷⁷Lu-HSA or ¹⁷⁷Lu-HSA-CDDP(4.1) via the tail vein. SPECT images were acquired at 10 min, 4 h, 24 h, 48 h and 72 h after injection. To acquire SPECT images, mice were anesthetized with 2% isoflurane and placed in the prone position. SPECT scans were acquired at 30 s per frame and 40 projections (frames) at an 18 angular step. The energy peaks of ¹⁷⁷Lu were set to 56.1 keV ±10%, 112.9 keV ±10%, and 208.4 keV±10%. Reconstructed data from SPECT were visualized using InVivoScope (Bioscan, Washington, D.C., USA).

### 1.13. Immunohistochemistry and TUNEL Assay

The tumor and kidney were fixed with 4% paraformaldehyde and embedded in paraffin, which was further cut into 4 m sections. To evaluate cellular apoptosis in kidney, a kidney section was stained using a TUNEL assay kit-HRP-DAB (ab206386; abcam, Cambridge, UK) according to the manufacturer's protocols.

### 1.14. Statistical Analysis

All statistical analyses were performed using GraphPad Prism. Student's t-test was used to determine the statistical significance of cellular uptake of HSA-CDDP, the antitumor effect of HSA-CDDP in the xenograft tumor model, and the biodistribution of HSA-CDDP in mice. P values below 0.05 were considered statistically significant.

### 2. Results

### 2.1. Characterization of HSA-CDDP

(1) In this study, we used CDDP-bound HSA as a therapeutic drug for tumors. Based on a previous paper, we set the CDDP binding condition to HSA. The molecular weight of HSA-CDDP was analyzed by the matrix-assisted laser desorption/ionization time of flight (MALDI-TOF). The molecular weight of HSA-CDDP was 67,522±129 Da. The proportion of CDDP per mole of HSA was calculated from the molecular weight difference between HSA-CDDP and HSA. The average number of molecules of CDDP bound to one mole of HSA was 4.1 (rounded up to the second digit after the decimal point). After we conjugated CDDP to HSA, we measured the molecular weight of HSA-CDDP(4.1) using MALDI-TOF.

To evaluate whether HSA-CDDP(4.1) can be taken up by glioblastoma cells in a SPARC-mediated HSA-dependent manner, we performed cellular uptake imaging using confocal microscopy based on our previous paper.

Two types of U87MG cell lines were used: U87MG cells, which highly express SPARC protein; U87MG-shSPARC cells, which exhibit low expression of SPARC protein.

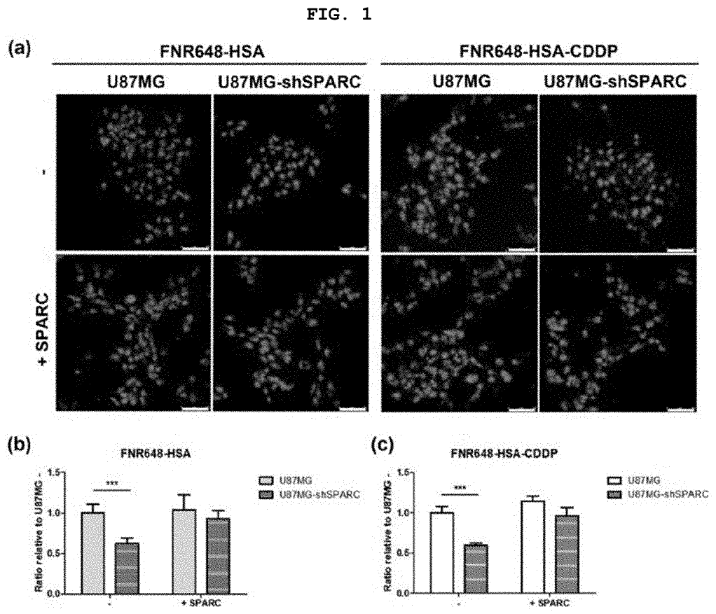

For objective comparison, the confocal microscopic images of cells were acquired and quantified (n=5 for each group). Our results showed that there is higher uptake of HSA-CDDP(4.1) in U87MG cells than U87MG-shSPARC cells ((a) of FIG. 1, (c) of FIG. 1, FNR648-HSA-CDDP, p<0.001).

To evaluate SPARC-mediated HSA-CDDP(4.1) uptake in cells, exogenous SPARC protein was co-treated in cells. By co-treatment of the exogenous SPARC in cells, HSA-CDDP(4.1) was highly accumulated in U87MG-shSPARC cells ((a) of FIG. 1, FNR648-HSA-CDDP in U87MG-shSPARC, and (c) of FIG. 1, +SPARC).

This SPARC-dependent uptake pattern was similar to the manner of HSA uptake in cells ((a) of FIG. 1, FNR648-HSA and FNR648-HSA-CDDP). This result demonstrated that the uptake of HSA-CDDP to cells was HSA dependent.

(2) For four (4) types of HSA-CDDPs prepared to evaluate cytotoxicity relative to the number of CDDPs bound to HSA, the average numbers of molecules of CDDPs bound to one mole of HSA were 1.5, 2.3, 5.4 and 7.7 moles, respectively.

### 2.2. Cytotoxic Effect of HA-CDDP In Vitro

### (1) Cytotoxic Effect of HSA-CDDP(4.1) In Vitro

The effect of HSA-CDDP(4.1) on cancer cell viability was also examined.

The cytotoxicity of HSA-CDDP(4.1) was studied in U87MG and U87MG-shSPAR cells using a cell counting kit-8 (CCK-8) assay.

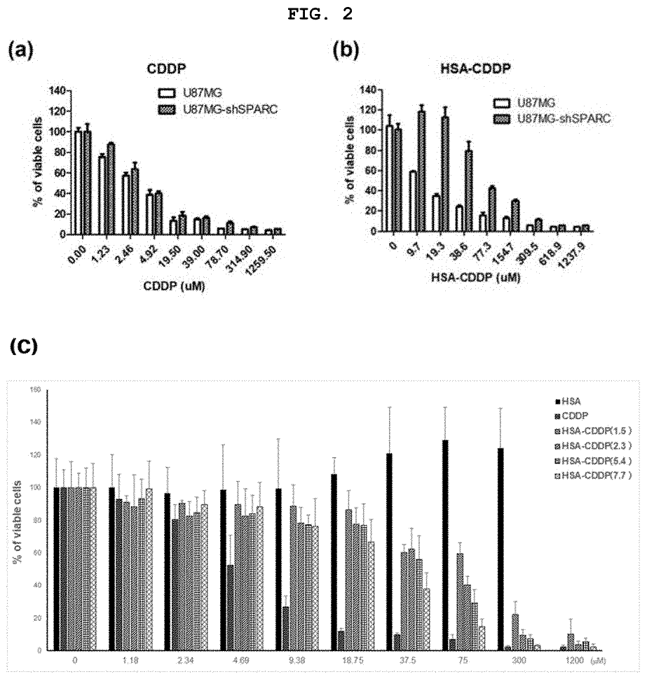

HSA-CDDP(4.1) showed dose-dependent toxicity toward cells ((b) of FIG. 2, HSA-CDDP). Specifically, the toxicity of HSA-CDDP(4.1) was higher with U87MG cells than U87MG-shSPARC cells ((b) of FIG. 2 and Table 1; IC50 for HSA-CDDP in two cells).

Since U87MG and U87MG-shSPARC showed similar cytotoxicity to CDDP ((a) of FIG. 2 and Table 1; IC50 for CDDP in cells), this indicates that the cytotoxicity of HSA-CDDP to U87MG and U87MG-shSPARC is dependent on the SPARC-mediated cellular uptake of HSA-CDDP

Table 1. IC50 values for CDDP and HSA-CDDP(4.1) in cells. Data are presented as means±SD (n=5).

TABLE 1

| IC₅₀ (μM) | U87MG | U87MG-shSPARC |
|---|---|---|
| CDDP | 2.545 ± 0.1345 | 2.646 ± 0.1302 |
| HSA-CDDP(4.1) | 11.49 ± 0.1726 | ~̅59.3 |

(2) Assessment of cytotoxicity to the number of CDDPs bound to HSA was executed. The cytotoxicity was studied in U87MG cells using the Cell Counting Kit-8 (CCK-8) assay. From the study, it was confirmed that toxicity became higher with increase in the number of CDDPs bound to HSA.

Table 2 below shows IC50 values for CDDP and CDDP number-dependent HSA. Data were indicated as mean SD (n=6).

TABLE 2

| IC₅₀ (μM) | U87MG |
|---|---|
| CDDP | 4.723 ± 0.1095 |
| HSA-CDDP(1.5) | ~̅100.7 |
| HSA-CDDP(2.3) | ~̅55.18 |
| HSA-CDDP(5.4) | 37.64 ± 0.1396 |
| HSA-CDDP(7.7) | 37.3 ± 0.1503 |

### (3) Apoptosis triggered by HSA-CDDP(4.1)

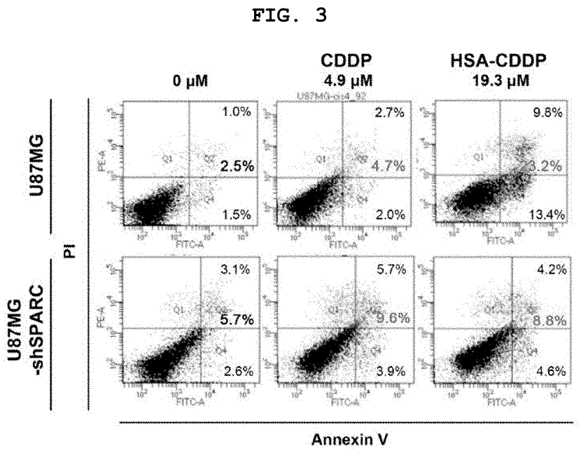

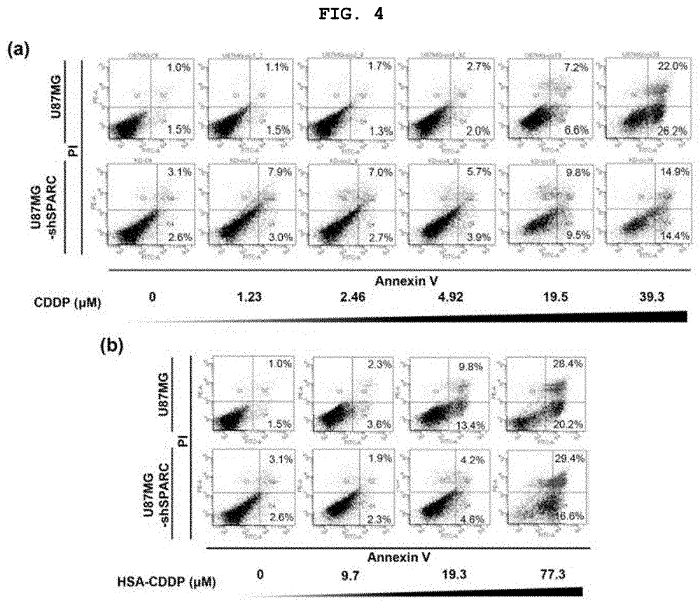

It is well known that cell apoptosis is the basic mechanism of action of CDDP. Apoptosis triggered by HSA-CDDP(4.1) was observed by flow cytometry analysis using AnnexinV-FITC/PI co-staining (FIG. 3 and FIG. 4). The result showed that HSA-CDDP induced apoptosis in a dose-dependent manner ((b) of FIG. 4).

We compared the percentage of apoptotic cells in the concentration of CDDP as 4.9 μM and HSA-CDDP(4.1) as 19.3 μM, which showed that 30-40% of cells were alive after drug treatment in cck-8 results.

U87MG exhibited a higher apoptosis rate than U87MG-shSPARC cells (FIG. 3, HSA-CDDP, 19.3 μM; U87MG, 23.2%; U87MG-shSPARC, 8.8%). Comparing CDDP 4.9 μand HSA-CDDP 19.3 μM in U87MG cells, HSA-CDDP exhibited a higher apoptotic cell percentage than CDDP, although they both showed similar cellular toxicity in CCK-8 (FIG. 3).

These results strongly indicate that the cellular toxicity of HSA-CDDP is SPARC-mediated, and HSA enhanced uptake in cells and apoptosis.

### 2.3. Antitumor Effect of HSA-CDDP In Vivo

The therapeutic effect of HSA-CDDP was investigated in a xenograft tumor mice model using U87MG and U87MG-shSPARC cells. Drug administration began when tumor size reached 50 mm³ and tumor growth was observed until the tumor volume reached 2000 mm³. Mice were administered PBS, CDDP, or HSA-CDDP(4.1) by intravenous injection every other day, to a total of seven times.

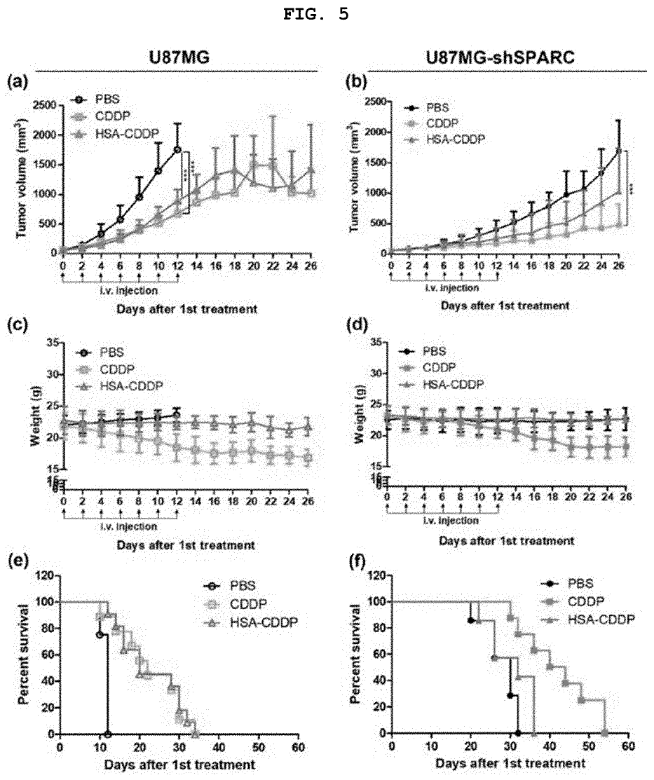

With regard to the U87MG tumor model, 12 days after the first drug treatment, the CDDP- and HSA-CDDP(4.1)-treated mice showed significantly reduced tumor growth than the PBS group ((a) of FIG. 5, p<0.001).

In the U87MG-shSPARC tumor model, 26 days after first drug treatment, CDDP-treated mice showed significantly reduced tumor growth than the PBS group ((b) of FIG. 5, p<0.001), but HSA-CDDP(4.1)-treated mice showed no difference in tumor growth to PBS. The weight of mice was significantly decreased in the CDDP-treated group ((c) of FIG. 5, (d) of FIG. 5), but this did not occur with HSA-CDDP(4.1)-treated mice. This means that CDDP may cause the negative effect in the in vivo system, but this effect was not seen in the HSA-CDDP treatment.

For the survival rate in the U87MG tumor model, CDDP- and HSA-CDDP(4.1)-treated mice exhibited a prolonged survival time than the PBS group ((e) of FIG. 5 and Table 3). In the U87MG-shSPARC tumor model, HSA-CDDP treated mice showed a similar survival rate to the PBS group, whereas CDDP-treated mice showed prolonged survival ((f) of FIG. 5, Table 3). These results strongly suggest that the antitumor effect of HSA-CDDP is based on the SPARC-mediated HSA-dependent uptake in tumors and is similar to CDDP.

Table 3. Median survival in days of U87MG and U87MG-shSPARC tumor xenograft model.

TABLE 3

| Median survival (Days) | PBS | CDDP | HSA-CDDP |
|---|---|---|---|
| U87MG | 12 | 22 | 20 |
| U87MG-shSPARC | 30 | 42 | 32 |

### 2.4. Biodistribution of HSA-CDDP In Vivo

The biodistribution of HSA-CDDP(4.1) was analyzed 72 h after the last HSA-CDDP treatment as an antitumor effect (treatment administered every other day seven times, beginning 15 days after the first HSA-CDDP(4.1) treatment) in normal mice and tumor-bearing mice.

The amount of CDDP in each organ (brain, heart, liver, kidney, spleen, lung, and blood) was measured using inductively coupled plasma mass spectrometry (ICP-MS).

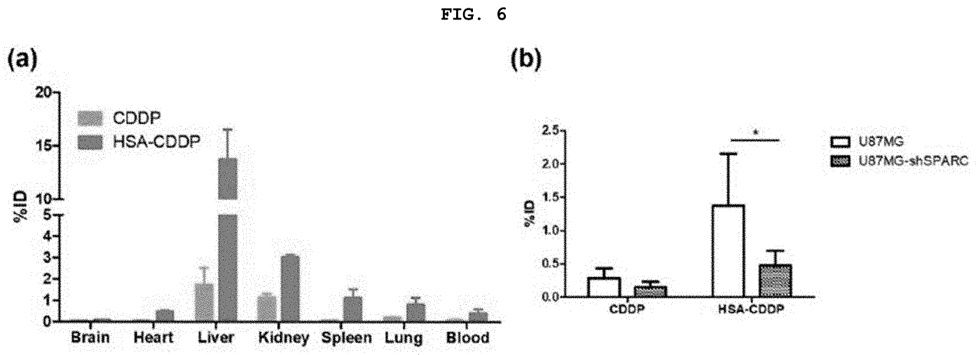

HSA-CDDP showed higher blood distribution than CDDP ((a) of FIG. 6). Due to the prolonged blood distribution, all organs except the brain showed higher accumulation of CDDP in the HSA-CDDP group. Particularly, HSA-CDDP was highly accumulated in the liver ((a) of FIG. 6).

In the U87MG tumor accumulation of CDDP data, the amount of CDDP in the HSA-CDDP group was significantly higher than the CDDP group ((b) of FIG. 6, p<0.05). In the HSA-CDDP group, U87MG tumors showed a higher accumulation of CDDP than U87MG-shSPARC tumors ((b) of FIG. 6, HSA-CDDP, p<0.05).

In the CDDP treatment group, there was no difference in CDDP accumulation between U87MG and U87MG-shSPARC ((b) of FIG. 6). In U87MG tumors, the HSA-CDDP group showed significantly increased CDDP accumulation than the CDDP group ((b) of FIG. 6).

The result shows that HSA-CDDP is taken up by tumors in an HSA-dependent manner.

### 2.5. Biosafety of HSA-CDDP In Vivo

In the antitumor effect result, mice treated with CCDP showed significant weight loss compared to the group treated with PBS. This result shows that there can be negative effects of the CDDP in vivo system, and this needs to be verified. It is well known that the major dose-limiting side effect of CDDP is nephrotoxicity.

In biodistribution studies, HSA-CDDP was highly accumulated in the liver. We assessed the toxicity of HSA-CDDP in the mice by monitoring the blood marker of liver, kidney function, and body weight 72 h after the final HSA-CDDP treatment as an antitumor effect plan (every other day, seven times, 15 days after the first HSA-CDDP treatment).

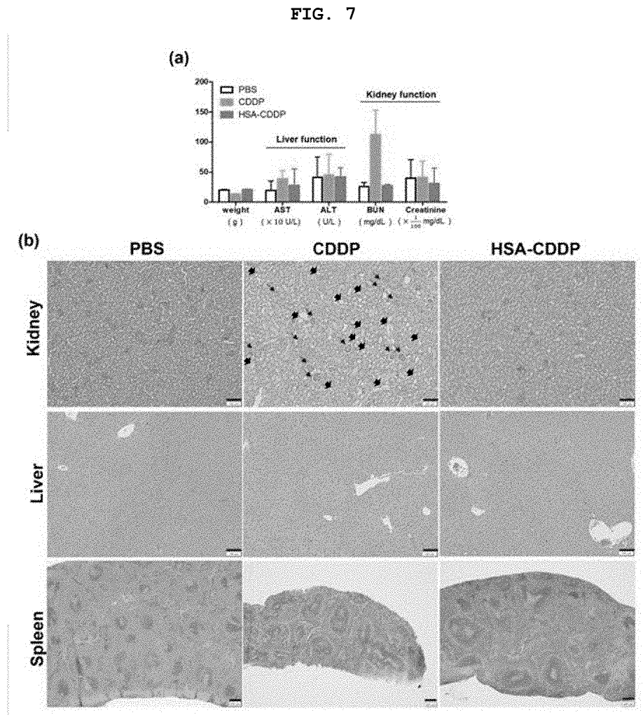

The CDDP treated group showed significant body weight loss compared to the PBS and HSA-CDDP groups, similar to the antitumor effect result for weight ((a) of FIG. 7).

In terms of liver function, aspartate transaminase (AST) and alanine aminotransferase (ALT) showed no significant difference among groups ((a) of FIG. 7).

For kidney function, blood urea nitrogen (BUN) increased in the CDDP group ((a) of FIG. 7). In the hematoxylin-eosin (H&E) staining images, CDDP treated kidney tissue showed tubular degeneration ((b) of FIG. 7, black arrows) and extensive epithelial vacuolization ((b) of FIG. 7, yellow arrows).

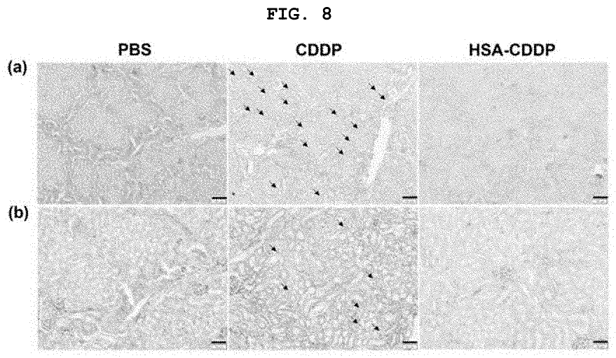

In the terminal deoxynucleotidyl transferase (TUNEL) assay, TUNEL positive cells were only identified in the kidney of CDDP-treated mice (FIG. 8).

From this result, it was confirmed that HSA-CDDP reduced the nephrotoxicity of CDDP in vivo.

1. A composition for treatment of a cancer expressing secreted protein acidic and rich in cysteine (SPARC), the composition comprising an albumin and at least one cisplatin bound thereto.

2. The composition of claim 1, wherein 1 to 8 cisplatins are bound to the albumin.

3. The composition of claim 1, wherein 4 to 5 cisplatins are bound to the albumin.

4. The composition for treatment of a cancer according to claim 3, wherein a conjugate of the albumin and the 4 to 5 cisplatins bound thereto has a molecular weight of 65,000 to 70,000 Da.

5. The composition for treatment of a cancer according to claim 3, wherein a conjugate of the albumin and the 4 to 5 cisplatins bound thereto has a size of 5 nm to 100 nm.

6. The composition for treatment of a cancer according to claim 1, wherein the cisplatin is bound to His side-chain residue or Met side-chain residue on a surface of the albumin.

7. The composition for treatment of a cancer according to claim 6, wherein His105 or His288 on the surface of the albumin is bound to the cisplatin.

8. The composition for treatment of a cancer according to claim 6, wherein Met298, Met329 or Met548 on the surface of the albumin is bound to the cisplatin.

9. The composition for treatment of a cancer according to claim 6, wherein the binding is obtained by a coordinate bond.

10. The composition for treatment of a cancer according to claim 1, wherein the cancer expressing SPARC is at least one selected from the group consisting of a brain tumor, melanoma, breast cancer, rectal cancer and stomach cancer.

11. The composition for treatment of a cancer according to claim 10, wherein the cancer is a brain tumor.

12. The composition for treatment of a cancer according to claim 11, the brain tumor is glioma.

13. The composition for treatment of a cancer according to claim 12, wherein the glioma is at least one selected from the group consisting of astrocytoma, glioblastoma and oligodendroblastoma.
